(19) United States
(12) Patent Application Publication
(10) Pub. No.: US 2026/0325877 A1
(43) Pub. Date: Oct. 22, 2026

(54) AUTOMATIC PLANNING METHOD, DEVICE AND SYSTEM FOR BRACHYTHERAPY BASED ON LARGE VISION-LANGUAGE MODEL, AND STORAGE MEDIUM

(71) Applicant: Fudan University Eye and ENT Hospital, Shanghai (CN)

(72) Inventors: Hongmeng Yu, Shanghai (CN); Yuxuan Shi, Shanghai (CN); Zhen Li, NewHaven, CT (US); Christopher J. Tien, Orange, CT (US); David J. Carlson, Madison, CT (US); Jiayu Lu, Boston, MA (US); Li Wang, Shanghai (CN); Xueli Liu, Shanghai (CN); Xiaofeng Liu, Waltham, MA (US)

(21) Appl. No.: 19/230,159

(22) Filed: Jun. 6, 2025

(30) Foreign Application Priority Data
Apr. 21, 2025 (CN) 202510500667.6

(51) Int. Cl. A61N 5/10; G16H 10/60; G16H 20/40

(52) U.S. Cl. CPC A61N 5/1031; A61N 5/1001; A61N 5/1038; G16H 10/60; G16H 20/40

(57) ABSTRACT

An automatic planning method, device and system for brachytherapy and a storage medium are provided. The method includes: acquiring an initial radiation plan file and a radiation structure file; preprocessing the initial radiation plan file and the radiation structure file to obtain preprocessed key data; inputting the preprocessed key data into a vision-language model to iteratively predict dwell times and dwell positions required by a brachytherapy plan through multiple optimization iterations; for each optimization iteration, calculating dose distribution according to dwell times and positions based on TG-43 formalism, comparing the dose metrics with predefined planning requirements, and feeding comparison results and control instructions back to the vision-language model to performing a next iteration until the dose metrics meet the predefined planning requirements, thereby yielding a finalized base plan; and refining and enhancing the finalized base plan to generate a target treatment plan tailored to personalized clinical needs of a patient.

### TECHNICAL FIELD

The disclosure relates to the field of brachytherapy technologies, and more particularly to an automatic planning method, an automatic planning device and an automatic planning system for brachytherapy based on large vision-language model, and a storage medium.

### BACKGROUND

High-dose rate brachytherapy (HDR-BT) is a specialized radiation therapy technique used to treat various types of cancer. It minimizes radiation exposure to surrounding healthy tissues by directly delivering high-dose radiation to the tumor site. HDR-BT is typically used as the primary treatment or in combination with other therapies, and has become a standard treatment for cancers that require high-dose radiation.

Current HDR-BT method relies on interactive (“manual”) treatment planning, which has limitations such as long-time consumption, large workload, lack of consistency due to variability between and within planners, plan quality relying on the experience and time investment of planners, and limited repeatability of plans. In addition, the treatment plan for HDR-BT faces enormous time pressure, as patients must remain fixed on a treatment bed and wait for the completion of the treatment plan before receiving treatment. Prolonged waiting can cause numbness in the patient’s legs, and involuntary movement can increase the risk of the applicator shifting or falling out of the patient's body. This time urgency forces medical physicists to complete treatment plans within a limited time frame, thereby increasing the risk of planning errors.

Automated HDR-BT treatment planning is an important solution to address the aforementioned challenges. In recent years, many studies have focused on automating the planning process. Significant progress has been made in automatic identification of the applicator and automatic segmentation of organs at risk (OARs) and high-risk clinical target volume (HRCTV), which improves efficiency and accuracy. In a process of plan generation and evaluation, some studies have explored the use of a deep learning neural network to predict dose-volume histograms (DVHs) and dose distribution maps. However, although these predictions provide valuable results, these methods cannot be directly applied to clinical practice since treatment machines require detailed dwell time and position information to accurately execute plans, and these studies only generate intermediate products of the plans without directly generating dwell times and dwell positions, thus unable to complete the automatic planning.

### SUMMARY

The technical problem to be solved of the disclosure is to provide an automatic planning method, an automatic planning device and an automatic planning system for brachytherapy and a storage medium.

In order to achieve the above purpose, the disclosure adopts the following technical solutions.

An automatic planning method for brachytherapy is provided, including:
step S1, acquiring an initial radiation plan file and a radiation structure file;
step S2, performing preprocessing on the initial radiation plan file and the radiation structure file to obtain preprocessed key data;
step S3, inputting the preprocessed key data into a vision-language model to iteratively predict dwell times and dwell positions required by a brachytherapy plan through multiple optimization iterations, where each of the dwell positions corresponds to one of the dwell times;
step S4, for each optimization iteration, calculating dose distribution based on the dwell times and the dwell positions using a formalism of Task Group No. 43 (TG-43) of American Association of Physicists in Medicine to obtain dose metrics, comparing the dose metrics with predefined planning requirements to obtain comparison results and control instructions, and feeding the comparison results and the control instructions back into the vision-language model to perform a next iteration until the dose metrics satisfy the predefined plan requirements, thereby yielding a finalized base plan; and

In an exemplary embodiment, the automatic planning method for brachytherapy further includes:

Specifically, before transmitting the target treatment plan to a treatment planning system for a physician to determine whether the target treatment plan is approved, the dwell times and the dwell positions are imported into the RP.dcm to obtain the target treatment plan.

In an embodiment, the step S2 includes:
step S21, obtaining the initial radiation plan file (RP.dcm) and the radiation structure file (RS.dcm) of the patient from a treatment planning system;
step S22, extracting a trajectory of a radiation source within an applicator and contours of OARs and HRCTV according to the radiation structure file; and
step s23, generating initial dwell positions and initial dwell times of each channel of the applicator according to the trajectory of the radiation source within the applicator.

In an embodiment, the step S3 includes:

The disclosure further provides an automatic planning device for brachytherapy, including a first processing module, a second processing module, a third processing module, a fourth processing module, and a fifth processing module.

The first processing module is configured to acquire an initial radiation plan file and a radiation structure file.

The second processing module is configured to perform preprocessing on the initial radiation plan file and the radiation structure file to obtain preprocessed key data.

The third processing module is configured to input the preprocessed key data into a vision-language model to iteratively predict dwell times and dwell positions required by a brachytherapy plan through multiple optimization iterations. Each of the dwell positions corresponds to one of the dwell times.

The fourth processing module is configured to: for each optimization iteration, calculate dose distribution based on the dwell times and the dwell positions using a formalism of TG-43 to obtain dose metrics, compare the dose metrics with predefined planning requirements to obtain comparison results and control instructions, and feed the comparison results and the control instructions back into the vision-language model to perform a next iteration until the dose metrics satisfy the predefined planning requirements, thereby yielding a finalized base plan.

The fifth processing module is configured to refine and enhance the finalized base plan to obtain a target treatment plan tailored to personalized clinical needs of a patient.

In an embodiment, the second processing module includes a first processing unit, a second processing unit, and a third processing unit.

The first processing unit is configured to obtain the initial RP.dcm and RS.dcm of this patient from the treatment planning system.

The second processing unit is configured to extract the trajectory of a radiation source within an applicator and contours of OARs and HRCTV from RS.dcm.

The third processing unit is configured to generate initial dwell positions and initial dwell times of each channel of the applicator according to the trajectory of the radiation source within the applicator.

In an embodiment, the third processing module is configured to input the predefined planning requirements, the initial dwell positions, the initial dwell times, the dose distribution map from last iteration and the contours of the OARs and the HRCTV into the large vision-language model, to obtain the optimized dwell times and the dwell positions required by the brachytherapy plan through the multiple iterations.

In an exemplary embodiment, each of the first processing module, the second processing module, the third processing module, the fourth processing module, the fifth processing module, the first processing unit, the second processing unit, the third processing unit and the large vision-language model is embodied by at least one processor and at least one memory coupled to the at least one processor, and the at least one memory stores computer programs executable by the at least one processor.

The disclosure further provides an automatic planning system for brachytherapy, including a memory and a processor. The memory is stored with a computer program executed by the processor, and the computer program is configured to, when executed by the processor, execute the automatic planning method for brachytherapy.

The disclosure further provides a non-transitory storage medium, the non-transitory storage medium is stored with a computer program, and the computer program is configured to execute the automatic planning method for brachytherapy during running.

The disclosure uses the large computer vision-language model to comprehensively analyze the image processing of patients, target volume position, and applicator position, and directly generate the dwell positions and dwell times required for planning, thereby completing the automatic planning for brachytherapy, reducing time for physicists to optimize plans, improving clinical efficiency, and enhancing the quality of plans.

### BRIEF DESCRIPTION OF DRAWINGS

In order to provide a clearer description of technical solutions in embodiments of the disclosure or in the related art, drawings required for the description of the embodiments or the related art will be briefly introduced below. Apparently, the drawings described below are merely some of the embodiments of the disclosure. For those skilled in the art, other drawings can be obtained based on the provided drawings without creative work.

FIG. 1 illustrates a flowchart of an automatic planning method for brachytherapy according to an embodiment of the disclosure.

FIG. 2 illustrates a flowchart of an automatic planning optimization iterative algorithm according to an embodiment of the disclosure.

FIG. 3 illustrates a schematic diagram of an automatic planning device for brachytherapy according to an embodiment of the disclosure.

FIG. 4 illustrates a schematic diagram of an automatic planning system for brachytherapy according to an embodiment of the disclosure.

FIG. 5 illustrates a schematic diagram of a non-transitory storage medium according to an embodiment of the disclosure.

### DETAILED DESCRIPTION OF EMBODIMENTS

The technical solutions in the embodiments of the disclosure will be clearly and completely described in conjunction with the accompanying drawings below. Apparently, the described embodiments are merely some of the embodiments of the disclosure, not all of them. Based on the embodiments of the disclosure, all other embodiments obtained by those skilled in the art without creative work are within a scope of protection of the disclosure.

In order to make the above purposes, features, and advantages of the disclosure more obvious and understandable, the disclosure is further described in detail in conjunction with the accompanying drawings and specific embodiments below.

### Embodiment 1

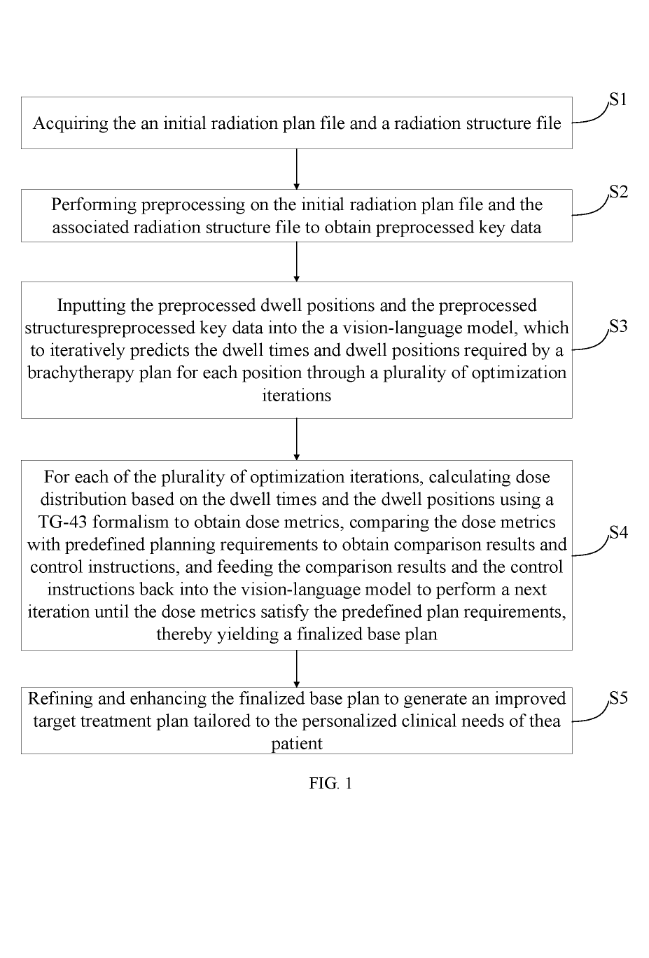

As shown in FIG. 1, the embodiments of the disclosure provide an automatic planning method for brachytherapy, including the following steps S1-S5.

In step S1, an initial radiation plan file (RP.dcm) and a radiation structure file (RS.dcm) of patient image data are acquired. Specifically, the initial radiation plan file (RP.dcm) and the radiation structure file (RS.dcm) are exported from the treatment planning system.

In step S2, the initial radiation plan file and the radiation structure file are preprocessed to obtain preprocessed key data.

In step S3, the preprocessed key data is input into a large vision-language model to iteratively predict dwell times and dwell positions required by a brachytherapy plan through multiple optimization iterations. Each of the dwell positions corresponds to one of the dwell times, and the dwell positions remain unchanged in each optimization iteration.

In step S4, for each optimization iteration, dose distribution is calculated based on the dwell times and the dwell positions using a formalism of TG-43 of American Association of Physicists in Medicine to obtain dose metrics. The dose metrics are compared with predefined planning requirements to obtain comparison results and control instructions. The comparison results and the control instructions are fed back into the large vision-language model to perform a next iteration until the dose metrics satisfy the predefined planning requirements, thereby yielding a finalized base plan. Specifically, the predefined planning requirements are the requirements of EMBRACE II (Image-guided intensity-modulated External Beam Radiochemotherapy and MRI-based adaptive Brachytherapy in locally advanced Cervical cancer) study protocol or institutional requirements.

In step S5, the base plan is refined and enhanced to obtain a target treatment plan tailored to personalized clinical needs of a patient.

In an embodiment of the disclosure, the step S2 includes the following steps S21-S23.

In step S21, the initial radiation plan file (RP.dcm) and the radiation structure file (RS.dcm) are obtained.

In step S22, a trajectory of a radiation source within an applicator and contours of OARs and HRCTV and specific coordinate positions of the contours of the OARs and the HRCTV in a space coordinate system are extracted according to the radiation structure file. All the OARs and HRCTV are plotted based on their z-axis coordinate, with different organs represented by different colors.

In step S23, initial dwell positions of each channel of the applicator are generated according to the trajectory of the radiation source within the applicator. Specifically, an interval distance between the dwell positions is any distance, for example, the interval distance is 1 millimeter (mm). The initial dwell times are set based on the initial dwell positions.

In an embodiment of the disclosure, in the step S3, the predefined planning requirements, the initial dwell positions, the initial dwell times, the dose distribution map from last iteration and the contours of the OARs and the HRCTV are input into the large vision-language model, to obtain the optimized dwell times and the dwell positions required by the brachytherapy plan through the multiple iterations. During optimization, dwell times are adjusted based on feedback from the previous iteration.

In an embodiment of the disclosure, in the step S4, the dwell positions and the dwell times are imported into a dose calculation program to automatically calculate the radiation dose distribution in the patient’s body, generate DHVs, dose-volume indexes (DVIs, i.e., the dose metrics) and dose distribution maps. In the brachytherapy plan, the dose distribution is calculated based on TG-43 consensus guideline, which refers to the recommendations on the dosimetry of interstitial brachytherapy sources published by the American Association of Physicists in Medicine (AAPM) in 1995.

In an embodiment, the parameters used for plan evaluation can be selected according to user-preference. The embodiment of the disclosure adopts the most commonly used D_{2cc}for the OARs, which represents the highest radiation dose of 2 cubic centimeters (cm³) (i.e. 2cc) in the OARs, and adopts the most commonly used D_{90%} for HRCTV, which represents a minimum dose received by 90% of the volume of the HRCTV. The number and type of the OARs can also be customized based on the brachytherapy plan requirements and institutional requirements. When the plan does not meet the plan requirement, the system will generate corresponding feedback and instructions to assist the large vision-language model in optimizing the plan. These feedbacks, as well as the dose distribution maps and DVIs from the previous iteration, are returned to the large vision-language model again. The large vision-language model then considers the necessary dwell time adjustments and generates a new dwell time for each dwell position to address issues such as excessive or insufficient dose distribution in OARs and HRCTV.

In an embodiment, during the entire process, all the images, text communication processes and transactive memory are continuously saved for 10 iterations, so that they can refer to the previous optimization history in subsequent iterations, and re-optimize the dwell times based on this. Once the plan meets the dose parameter requirements for all HRCTV and OARs, the final dwell times and dwell positions will be exported to the treatment machine for plan implementation.

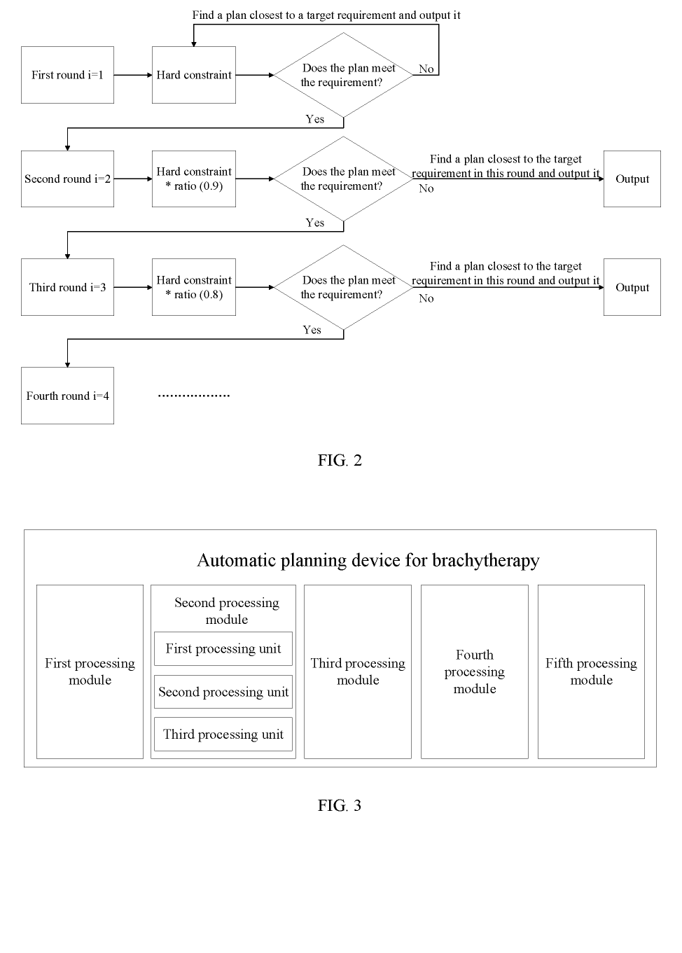

In an embodiment, in the step S4, in order to obtain a more reasonable and optimized dose distribution, to ensure that OARs are less affected by dose and to ensure that the HRCTV receive sufficient dose. As shown in FIG. 2, the embodiment of the disclosure proposes an automatic planning optimization iteration algorithm, which adopts a round-by-round optimization strategy to improve the feasibility of optimization and ensure the quality of the final solution by gradually relaxing hard constraints. Specifically, the hard constraints refer to mandatory planning requirements that adhere to established guidelines, such as EMBRACE II. These guidelines are widely adopted by most medical centers. Specifically, EMBRACE II recommends that for the total treatment course: the D_{90%} of the HRCTV should receive at least 85 Gy EQD2; the D_{2cc} of the bladder should be less than 90 Gy; the D_{2cc} of the rectum and sigmoid should each be less than 75 Gy; and the D_{2cc} of the small bowel should also remain below 75 Gy. These constraints can be further customized according to individual or institutional protocols.

In the first round of optimization, initial hard constraints (i.e., the predefined planning requirements * 100%) is used for optimization, and whether the optimization objectives are successfully met is determined. When the optimization objectives are met, proceed to the next round of optimization and further adjust the constraints to find a better solution. Then, enter the second round of optimization, the hard constraints are adjusted to 90% of the initial hard constraints and re-optimize. When the optimization objectives are met in the second round, enter the third round of optimization, the hard constraints are adjusted to 80% of the initial hard constraints and optimize again. During each optimization round, when the optimization is successful (i.e., the optimization objectives are met), it will enter the next round of optimization to continuously improve the plan. When the optimization fails (i.e., the optimization objectives are not met) after 20 iterations, the optimization process terminates and the optimal solution is selected from the 20 iterations in the current round as the final output. This method ensures that the hard constraints are satisfied as much as possible during the optimization process, while allowing for appropriate strategy adjustments when necessary to achieve the optimal solution for the specific patient. At the same time, when users tend to prioritize the protection of one specific OAR for patient during the optimization process, the bladder is taken as an example, the optimization goal of the bladder is adjusted to 90%, 80%, and 70% of the initial hard constraint in each round for optimization until the optimization cannot be completed and stops.

In an embodiment of the disclosure, in order to meet the needs of users, the system can customize plans according to the physician’s requirements. When further optimizing a base plan that has already met the conditions, a plan can be developed based on the patient's condition. When an OAR (taking the bladder as an example) of the patient needs special protection, the optimization weight can be tilted towards the bladder while meeting the base plan requirements to ensure that the bladder receives minimal doses. The step S4 is to generate the base plan (for example, all OARs receive physical doses less than 4 Gy), and the base plan should meet the basic requirements of EMBRACE II study protocol for patient protection. The step S5 is to further tighten the optimization conditions based on the step S4 and generate the optimal plan (i.e., the target plan). Specifically, the step S5 has two options (i.e., the optimization objectives): 1. all organ dose protections are increased by 20%; and 2. according to the patient's personal situation, it is particularly important to protect a certain organ (such as the bladder), and the bladder protection should be increased to 40% separately.

A specific process of the automatic planning method for brachytherapy of the embodiment of the disclosure includes the follows.

### 1. Data preprocess

The position of applicator is identified, and corresponding dwell positions are generated according to a feasible trajectory of the radiation source within the applicator.

Read RS.dcm data to extract position information of the HRCTV, the OARs and the applicator.

Two-dimensional positional images are generated for each slice, in which the relative positions of the HRCTV, OARs, and dwell positions are visualized through image fusion. Each slice yields a 2D image, and these images are sequenced according to the slice's z-coordinate.

### 2. Data input

All key data is input into the large vision-language model, so that it has comprehensive treatment information and supports optimization and iteration.

Positional images related to the dwell positions, HRCTV, and the OARs, the dwell positions, the dwell times, the requirement of the radiation plan, hard constraints, and the dose distribution maps, generated during the previous optimization process are input into the large vision-language model.

The input information is stored through a memory module while controlling the amount of stored information, only storing information within 10 iterations to avoid information explosion.

When it is found that the plan does not meet the requirement after evaluation by an evaluation module, feedback information related to the plan will be automatically generated as data input. The feedback information is a specific instruction in a form of words, such as “the current D₉₀ _{%} of the HRCTVs is xxx, the dose of the requirement of the radiation plan is xxx, the current D_{2cc} of the bladder is xxx, and the dose of the requirement of the radiation plan is xxx; and the current patient dose distribution is non-conformable (pear shaped distribution in cervical cancer is taken as an example), and please adjust the dwell time”, which are input into the large vision-language model again with other data.

### 3. Data output

The large vision-language model evaluates the input information (including the feedback for the previous plan, the dose distribution maps of different slices in the previous plan, and a relative distribution map among the HRCTV, the OARs and the dwell positions), and areas need to improve dose or decrease dose and come up with a solution to meet the requirement. The adjustment strategy is to comprehensively consider the dose coverage of the HRCTVs and the protection of the OARs. The dose needs to meet the requirement of the radiation plan, while also ensuring that the patient's dose distribution is pear shaped.

After evaluation, the large vision-language model generates a new and optimized dwell times according to its own understanding and adjustment strategy of the task, and the dwell times will be calculated together for the dose distribution. The dose calculation model based on TG-43 formula is adopted for calculation, to obtain new dose metrics (DVIs are D_{2cc} and D_{90%}), DVHs and dose distribution maps of the HRCTV and OARs.

### 4. Plan evaluation

The planning evaluation module evaluates the new generated plan (cervical cancer is taken as an example). The evaluation contents include that the dose distribution maps of the generated plan is evaluated to check whether it conforms to the pear-shaped dose distribution; whether the key dose metrics (D_{2cc} and D_{90%}) meet the requirement of the radiation plan is evaluated; whether the dwell time distribution of the implantation needle meets the requirement of the radiation plan is evaluated; and whether the dose distribution is smooth and excessive, without causing significant dose cold and hot zones is evaluated. When the evaluation is not qualified, continue to optimize the plan and generate a new treatment plan.

### 5. Plan Improvement

After the base plan that meets the basic requirement is generated, the base plan is further optimized and iterated. The optimization methods include the follows. 1. The indexes of all OARs are further tightened, and all organs are simultaneously optimized. 2. According to the physician's preference for OAR protection, the OARs that need to be protected first are given the highest protection weight, so as to obtain more suitable planning protection than the base plan.

### 6. Plan transmission

After the final plan selection and evaluation are completed, the system will automatically transmit the dwell positions and the dwell times to the brachytherapy afterloader for patient treatment.

As shown in FIG. 3, the embodiments of the disclosure further provide an automatic planning device for brachytherapy, including a first processing module, a second processing module, a third processing module, a fourth processing module, and a fifth processing module.

The first processing module is configured to acquire an initial radiation plan file and a radiation structure file.

The second processing module is configured to perform preprocessing on the initial radiation plan file and the radiation structure file to obtain preprocessed key data.

The third processing module is configured to input the preprocessed key data into a vision-language model to iteratively predict dwell times and dwell positions required by a brachytherapy plan through multiple optimization iterations. Each of the dwell positions corresponds to one of the dwell times.

The fourth processing module is configured to: for each optimization iteration, calculate dose distribution based on the dwell times and the dwell positions using a formalism of TG-43, compare the dose metrics with predefined planning requirements to obtain comparison results and control instructions, and feed the comparison results and the control instructions back into the vision-language model to perform a next iteration until the dose metrics satisfy the predefined planning requirements, thereby yielding a finalized base plan.

The fifth processing module is configured to refine and enhance the finalized base plan to obtain a target treatment plan tailored to personalized clinical needs of a patient.

In an embodiment of the disclosure, the second processing module includes a first processing unit, a second processing unit, and a third processing unit.

The first processing unit is configured to obtain the RP.dcm and RS.dcm of this patient from the treatment planning system.

The second processing unit is configured to extract the trajectory of a radiation source within an applicator and contours of OARs and HRCTV from RS.dcm.

The third processing unit is configured to generate initial dwell positions and initial dwell times of each channel of the applicator according to the trajectory of the radiation source within the applicator.

In an embodiment of the disclosure, the third processing module is configured to input the predefined planning requirements, the initial dwell positions, the initial dwell times, the dose distribution map from last iteration and the contours of the OARs and the HRCTV into the large vision-language model, to obtain the optimized dwell times and the dwell positions required by the brachytherapy plan through the multiple iterations.

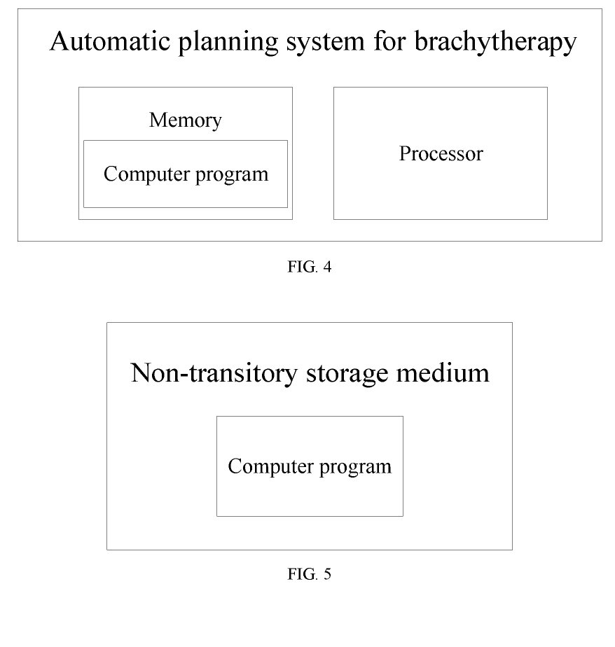

As shown in FIG. 4, the embodiments of the disclosure further provide an automatic planning system for brachytherapy, including a memory and a processor. The memory is stored with a computer program executed by the processor, and the computer program is configured to, when executed by the processor, execute the automatic planning method for brachytherapy.

As shown in FIG. 5, the embodiments of the disclosure further provide a non-transitory storage medium, the non-transitory storage medium is stored with a computer program, and the computer program is configured to execute the automatic planning method for brachytherapy during running.

The above embodiments are merely descriptions of some of the embodiments of the disclosure and do not limit a scope of the disclosure. Various modifications and improvements made by those skilled in the art to the technical solution of the disclosure without departing from a design spirit of the disclosure should fall within the scope of protection determined by claims of the disclosure.

What is claimed is:

1. An automatic planning method for brachytherapy, comprising:
step S1, acquiring an initial radiation plan file and a radiation structure file;
step S2, performing preprocessing on the initial radiation plan file and the radiation structure file to obtain preprocessed key data;
step S3, inputting the preprocessed key data into a vision-language model to iteratively predict dwell times and dwell positions required by a brachytherapy plan through a plurality of optimization iterations, wherein each of the dwell positions corresponds to one of the dwell times;
step S4, for each of the plurality of optimization iterations, calculating dose distribution based on the dwell times and the dwell positions using a formalism of Task Group No. 43 (TG-43) of American Association of Physicists in Medicine to obtain dose metrics, comparing the dose metrics with predefined planning requirements to obtain comparison results and control instructions, and feeding the comparison results and the control instructions back into the vision-language model to perform a next iteration until the dose metrics satisfy the predefined plan requirements, thereby yielding a finalized base plan; and
step S5, refining and enhancing the finalized base plan to generate a target treatment plan tailored to personalized clinical needs of a patient.

2. The automatic planning method for brachytherapy as claimed in claim 1, wherein the step S2 comprises:
step S21, obtaining the initial radiation plan file and the radiation structure file;
step S22, extracting a trajectory of a radiation source within an applicator and contours of organs at risk and clinical target volume according to the radiation structure file; and
step S23, generating initial dwell positions and initial dwell times of each channel of the applicator according to the trajectory of the radiation source within the applicator.

3. The automatic planning method for brachytherapy as claimed in claim 2, wherein the step S3 comprises:
inputting the predefined planning requirements, the initial dwell positions, the initial dwell times, a dose distribution map from last iteration and the contours of the organs at risk and the clinical target volume into the vision-language model, to obtain the dwell times and the dwell positions required by the brachytherapy plan through the plurality of iterations.

4. An automatic planning device for brachytherapy, comprising:
a first processing module, configured to acquire an initial radiation plan file and a radiation structure file;
a second processing module, configured to perform preprocessing on the initial radiation plan file and the radiation structure file to obtain preprocessed key data;
a third processing module, configured to input the preprocessed key data into a vision-language model to iteratively predict dwell times and dwell positions required by a brachytherapy plan through a plurality of optimization iterations, wherein each of the dwell positions corresponds to one of the dwell times;
a fourth processing module, configured to: for each of the plurality of optimization iterations, calculate dose distribution based on the dwell times and the dwell positions using a formalism of TG-43 to obtain dose metrics, compare the dose metrics with predefined planning requirements to obtain comparison results and control instructions, and feed the comparison results and the control instructions back into the vision-language model to perform a next iteration until the dose metrics satisfy the predefined planning requirements, thereby yielding a finalized base plan; and
a fifth processing module, configured to refine and enhance the finalized base plan to obtain a target treatment plan tailored to personalized clinical needs of a patient.

5. The automatic planning device for brachytherapy as claimed in claim 4, wherein the second processing module comprises:
a first processing unit, configured to obtain the initial radiation plan file and the radiation structure file;
a second processing unit, configured to extract a trajectory of a radiation source within an applicator and contours of organs at risk and clinical target volume according to the radiation structure file; and
a third processing unit, configured to generate initial dwell positions and initial dwell times of each channel of the applicator according to the trajectory of the radiation source within the applicator.

6. The automatic planning device for brachytherapy as claimed in claim 5, wherein the third processing module is configured to input the predefined planning requirements, the initial dwell positions, the initial dwell times, a dose distribution map from last iteration and the contours of the organs at risk and the clinical target volume into the vision-language model, to obtain the dwell times and the dwell positions required by the brachytherapy plan through the plurality of iterations.

7. An automatic planning system for brachytherapy, comprising: a memory and a processor, wherein the memory is stored with a computer program executed by the processor, and the computer program is configured to, when executed by the processor, execute the automatic planning method for brachytherapy as claimed in claim 1.

8. A non-transitory storage medium, wherein the non-transitory storage medium is stored with a computer program, and the computer program is configured to execute the automatic planning method for brachytherapy as claimed in claim 1 during running.
